SEQUENCE LISTING (18 sequences)

SEQ ID NO: 1 - DNA, 3604 bp, Homo Sapiens
ataacgcaggaaagaacatgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacctcgaccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgggcccgcgagccaggccggcgcttctcctcctgatgcttttgcagaccgcggtcctgcaggggcgcttgctgcgttcacactctctgcactacctcttcatgggtgcctcagagcaggaccttggtctttccttgtttgaagctttgggctacgtggatgaccagctgttcgtgttctatgatcatgagagtcgccgtgtggagccccgaactccatgggtttccagtagaatttcaagccagatgtggctgcagctgagtcagagtctgaaagggtgggatcacatgttcactgttgacttctggactattatggaaaatcacaaccacagcaaggagtcccacaccctgcaggtcatcctgggctgtgaaatgcaagaagacaacagtactgagggctactggaagtacgggtatgatgggcaggaccaccttgaattctgccctgacacactggattggagagcagcagaacccagggcctggcccaccaagctggagtgggaaaggcacaagattcgggccaggcagaacagggcctacctggagagggactgccctgcacagctgcagcagttgctggagctggggagaggtgttttggaccaacaagtgcctcctttggtgaaggtgacacatcatgtgacctcttcagtgaccactctacggtgtcgggccttgaactactacccccagaacatcaccatgaagtggctgaaggataagcagccaatggatgccaaggagttcgaacctaaagacgtattgcccaatggggatgggacctaccagggctggataaccttggctgtaccccctggggaagagcagagatatacgtgccaggtggagcacccaggcctggatcagcccctcattgtgatctgggagccctcaccgtctggcaccctagtcattggagtcatcagtggaattgctgtttttgtcgtcatcttgttcattggaattttgttcataatattaaggaagaggcagggttcaagaggagccatggggcactacgtcttagctgaacgtgagtgaggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatgg

SEQ ID NO: 2 - DNA, 42 bp, Artificial Sequence
  Oligonucleotide UCori-Bam
ggatccagatctactcgaggtagaaaagatcaaaggatcttc

SEQ ID NO: 3 - DNA, 31 bp, Artificial Sequence
  Oligonucleotide UCori-Nco
agtccatggataacgcaggaaagaacatgtg

SEQ ID NO: 4 - DNA, 31 bp, Artificial Sequence
  Oligonucleotide hGH-F
aggatccgaattccctgtgacccctccccag

SEQ ID NO: 5 - DNA, 40 bp, Artificial Sequence
  Oligonucleotide hGH-R
ctctttaccaattctacgcgtaaggacagggaagggagca

SEQ ID NO: 6 - DNA, 40 bp, Artificial Sequence
  Oligonucleotide RO-F
cttccctgtccttacgcgtagaattggtaaagagagtcgt

SEQ ID NO: 7 - DNA, 41 bp, Artificial Sequence
  Oligonucleotide RO-R
ccgtagaaaactagttaatgatttttatcaaaatcattaag

SEQ ID NO: 8 - DNA, 49 bp, Artificial Sequence
  Oligonucleotide RO-1
gaattggtaaagagagtcgtgtaaaatatcgagttcgcacatcttgttg

SEQ ID NO: 9 - DNA, 47 bp, Artificial Sequence
  Oligonucleotide RO-2
gatttttggcgaaaccatttgatcatatgacaagatgtgtatctacc

SEQ ID NO: 10 - DNA, 47 bp, Artificial Sequence
  Oligonucleotide RO-3
atgatttttatcaaaatcattaagttaaggtagatacacatcttgtc

SEQ ID NO: 11 - DNA, 34 bp, Artificial Sequence
  Oligonucleotide Kan-F
aaatcattaactagttttctacggggtctgacgc

SEQ ID NO: 12 - DNA, 33 bp, Artificial Sequence
  Oligonucleotide Kan-R
cagccatggactagtggtggcacttttcgggga

SEQ ID NO: 13 - DNA, 30 bp, Artificial Sequence
  Oligonucleotide MCS1
gatccgatatcgtcgacaagcttggtacct

SEQ ID NO: 14 - DNA, 20 bp, Artificial Sequence
  Oligonucleotide MCS2
caagcttgtcgacgatatcg

SEQ ID NO: 15 - DNA, 32 bp, Artificial Sequence
  Oligonucleotide MCS3
ccggagcggccgctctagagctagcgacgtcg

SEQ ID NO: 16 - DNA, 42 bp, Artificial Sequence
  Oligonucleotide MCS4
aattcgacgtcgctagctctagagcggccgctccggaggtac

SEQ ID NO: 17 - DNA, 25 bp, Artificial Sequence
  Oligonucleotide EF1-Xho
atctcgagcgtgaggctccggtgcc

SEQ ID NO: 18 - DNA, 29 bp, Artificial Sequence
  Oligonucleotide EF1-R
tcggatcctggcttttaggggtagttttc

(19) United States
(12) Patent Application Publication
(10) Pub. No.: US 2025/0195690 A1
(43) Pub. Date: Jun. 19, 2025

(54) Gene therapy DNA vector based on gene therapy DNA vector GDTT1.8NAS12 carrying HFE therapeutic gene for enhanced expression of the therapeutic gene, method of its production and use, Escherichia coli strain JM110-NAS/GDTT1.8NAS12-HFE carrying the gene therapy DNA vector, method of its production, method of the gene therapy DNA vector production on an industrial scale

(71) Applicants: GENETIC DIAGNOSTICS AND THERAPY 21 LTD, London (GB); OBSCHESTVO S OGRANICHENNOI OTVETSTVENNOSTJU "REKOMBITEKH", Moskva (RU)

(72) Inventor: Anton GAMOLSKI, London (GB)

(21) Appl. No.: 18/721,291

(22) PCT Filed: Feb. 20, 2021
(86) PCT No.: PCT/RU2021/000074

§ 371 Date: Jun. 18, 2024

(30) Foreign Application Priority Data
Dec. 30, 2019 (RU) 2019145031

(51) Int. Cl. A61K 48/00; A61K 35/12; A61K 38/17; A61P 3/00; C12N 1/20; C12N 15/10; C12N 15/70; C12N 15/85; C12R 1/19

(52) U.S. Cl. CPC A61K 48/005; A61K 35/12; A61K 38/1774; A61P 3/00; C12N 1/20; C12N 15/101; C12N 15/1017; C12N 15/70; C12N 15/85; C12N 2800/24; C12R 2001/19

(57) ABSTRACT

Proposed is a gene-therapy DNA vector based on gene-therapy DNA vector GDTT1.8NAS12 for treating diseases characterized by impaired functioning of the HFE protein responsible for regulating iron metabolism in the human body, and for treating diseases related to impaired expression of the HFE gene, inter alia diseases caused by insufficient expression of the HFE gene and/or by the presence of mutations in the HFE gene, inter alia in the case of haemochromatosis, wherein the gene-therapy DNA vector contains the coding part of the target gene HFE, cloned in gene-therapy DNA vector GDTT1.8NAS12 to produce gene-therapy DNA vector GDTT1.8NAS12-HFE having the nucleotide sequence SEQ ID NO: 1. The resulting gene-therapy DNA vector GDTT1.8NAS12-HFE is capable of effectively penetrating into human and animal cells and expressing the target gene cloned in it, i.e. HFE, by virtue of the limited size of the vector part GDTT1.8NAS12, which is not greater than 2600 bp. In the proposed gene-therapy DNA vector GDTT1.8NAS12-HFE, nucleotide sequences which are not antibiotic resistance genes, viral genes or regulatory elements of viral genomes are used as structural elements, thus allowing the gene-therapy DNA vector to be used safely for gene therapy in humans and animals.

### TECHNICAL FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and subsequent translation of protein molecules encoded by these genes. Within the description of the invention, gene expression refers to the production of a protein molecule with amino acid sequence encoded by this gene.

HFE gene plays a key role in several processes in human and animal organisms. The HFE gene encodes the HFE protein involved in iron metabolism regulation in humans. Mutations in the HFE gene are the main cause of haemochromatosis, a hereditary disease that manifests as an excessive iron accumulation in internal organs and skin.

HFE protein consists of several domains: a signal peptide; an extracellular region binding to a transferrin receptor; a domain with a structure similar to an immunoglobulin molecule; a transmembrane domain anchoring the protein to the cell membrane; and a short cytoplasmic region. The HFE protein is found in liver, intestine, and immune system cells. Its main function is to regulate the iron metabolism in a body (Feder et al., 1996).

To block iron transport into a cell, HFE protein binds to the transferrin receptor 1 (TFR1) prohibiting further interaction of the receptor with the transferrin (TF) protein that transports atoms of iron (Feder et al., 1998). HFE protein regulates production of the hepcidin hormone. Hepcidin is synthesized in liver and is responsible for iron uptake from food and iron release from storage. When HFE protein is not bound to a TFR1 receptor, it serves as a key element in the cascade inducing hepcidin expression (Nemeth et al., 2006).

The connection between low concentration of functional HFE protein and various adverse conditions in humans has been demonstrated. Thus, hereditary haemochromatosis (type I haemochromatosis), with a prevalence of 1.5-3 per 1000 persons, is caused by mutations in the HFE gene localized in chromosome 6: C282Y and H63D, in 95% of cases. Type I haemochromatosis is a disease manifested in the form of iron accumulation in parenchymal organs (predominantly in liver, pancreas, heart, and joint) with collateral damage of those organs due to free radical production (Barton et al., 2006).

The classical disease triad includes cirrhosis, diabetes mellitus, and skin pigmentation disorder in mid-aged people, predominantly in men. The most common complication is hepatic disorder that can progress to cirrhosis; 20-30% of cirrhosis patients develop hepatocellular carcinoma. Hepatic disorders are among the most common causes of death. The second most common cause of death is cardiomyopathy with cardiac insufficiency. Hyperpigmentation (bronze diabetes) is a general symptom, as well as secondary arthropathy.

Thus, the increase of expression of the functional HFE gene via gene therapy has potential to correct various conditions in humans and animals.

Analysis of approaches to increase the expression of therapeutic genes implies the practicability of use of different gene therapy vectors.

Gene therapy vectors are divided into viral, cell, and DNA vectors (Guideline on the quality, non-clinical, and clinical aspects of gene therapy medicinal Products EMA/CAT/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N. // Expert Rev Vaccines. 2016; 15(3):313-29).

However, limitations of plasmid vectors use in gene therapy include: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression level of therapeutic genes (promoters, enhancers, post-translational regulatory elements) that constitute nucleotide sequences of genomes of various viruses (Draft Guideline on the quality, non-clinical and clinical aspects of gene therapy medicinal products, http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R. // Mol Biotechnol. 2008.39(2):97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HELA cells with 383-4548 bp DNA vectors it was shown that the difference in penetration efficiency can be up to two orders of magnitude (100 times different) (Hornstein B D et al. //PLoS ONE. 2016; 11(12): e0167537).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2). The vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

The following applications served as prototypes of this invention with regard to the use of gene therapy approaches to increase the expression level of the HFE gene.

Patent RU2678756 describes gene therapy DNA vector VTvaf17, method of its production; Escherichia coli strain SCS110-AF, method of its production; Escherichia coli strain SCS110-AF/VTvaf17 carrying the gene therapy DNA vector VTvaf17, method of its production. The disadvantage of this invention is the considerable length of the vector part, which may have an adverse effect on the efficiency of DNA vector delivery to cells.

A method of DNA vector expressing HFE gene is known. Vectors based on pcDNA3.1 plasmid were obtained and introduced into HeLa cell line by lipofection (Gross C N, Irrinki A, Feder J N, Enns C A). Co-trafficking of HFE, a nonclassical major histocompatibility complex class I protein, with the transferrin receptor implies a role in intracellular iron regulation. J Biol Chem. 1998 273(34):22068-74). The use of this DNA vector in gene therapy is limited by the presence of regulatory sequences of viral genomes.

We are aware of a patent (U.S. Pat. No. 7,078,513 B1) describing HFE gene expression in heterologous and homologous systems. The patent provides examples of adenoviral and retroviral vector obtainment for in vivo gene therapy. The disadvantage of adenoviral vectors is a time limit to therapeutic gene expression and induction of an immune response to repeated introduction of the vectors. The disadvantage of retroviral vectors is a potential induction of carcinogenesis.

There is an official letter on the application US 20190076551 describing a method of hereditary haemochromatosis treatment using a genome editing technology. A method is described for introduction of expressing structures into cells to encode nuclease genes and direct RNA to make double-strand breaks in the HFE gene in order to restore its function. This method has a disadvantage of potential off-target effects, i.e. making breaks in various non-targeted DNA sequences in the genome due to non-specific action of the enzyme.

### SUMMARY OF THE INVENTION

The purpose of this invention is to construct the gene therapy DNA vector for enhancement of the HFE gene expression in human and animal cells, combining the following properties:

I) Efficiency of the gene therapy DNA vector in order to increase the expression level of the therapeutic genes in eukaryotic cells.
II) Possibility of safe use in gene therapy of human beings and animals due to the absence of regulatory elements representing the nucleotide sequences of viral genomes in the gene therapy DNA vector.
III) Possibility of safe use in the gene therapy of human beings and animals due to the absence of antibiotic resistance genes in the gene therapy DNA vector.
IV) Producibility and constructability of the gene therapy DNA vector on an industrial scale.

Items II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes construction of a strain carrying this gene therapy DNA vector for the development and production of this gene therapy DNA vector on an industrial scale.

The specified purpose is achieved by construction of a gene therapy DNA vector based on the gene therapy DNA vector GDTT1.8NAS12 in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis, while the gene therapy DNA vector GDTT1.8NAS12-HFE contains the coding region of the HFE therapeutic gene cloned into the gene therapy DNA vector GDTT1.8NAS12 with the nucleotide sequence SEQ ID No. 1.

Due to the limited size of GDTT1.8NAS12 vector part not exceeding 2600 bp, the constructed gene therapy DNA vector GDTT1.8NAS12-HFE, has the ability to efficiently penetrate into human and animal cells and express the HFE therapeutic gene cloned to it.

The obtained gene therapy DNA vector GDTT1.8NAS12-HFE uses nucleotide sequences that don't constitute antibiotic resistance genes, viral genes, or regulatory elements of viral genomes, which ensures its safe use for gene therapy in humans and animals.

A method of gene therapy DNA vector production based on the gene therapy DNA vector GDTT1.8NAS12 carrying the therapeutic gene HFE has also been developed that involves obtaining the gene therapy DNA vector GDTT1.8NAS12-HFE as follows: the coding region of the HFE therapeutic gene is cloned to the gene therapy DNA vector GDTT1.8NAS12, and the gene therapy DNA vector GDTT1.8NAS12-HFE, SEQ ID No. 1 is obtained, while the coding region of the HFE therapeutic gene is obtained by isolating total RNA from the biological human tissue sample followed by the reverse transcription reaction and PCR amplification using the obtained oligonucleotides and cleaving the amplification product by corresponding restriction endonucleases, while cloning to the gene therapy DNA vector GDTT1.8NAS12 is performed by SalI and KpnI restriction sites, while the selection is performed without antibiotics, the following oligonucleotides produced for this purpose are used as dedicated oligonucleotides during the production of the gene therapy DNA vector GDTT1.8NAS12-HFE, SEQ ID No. 1 for the reverse transcription and PCR amplification:

HFE-up
TTTGTCGACCACCATGGGCCCGCGAGCCAGGCCGG,
HFE-lo
AATGGTACCTCACTCACGTTCAGCTAAGACGTAGTGC,

and the cleaving of the amplification product and cloning of the coding region of the HFE gene to the gene therapy DNA vector GDTT1.8NAS12 is performed with using SalI and KpnI restriction endonucleases.

A method of use of the gene therapy DNA vector based on the gene therapy DNA vector GDTT1.8NAS12 carrying the HFE therapeutic gene in order to treat diseases associated with disorders of functions of HFE protein responsible for regulation of iron metabolism in humans, in order to treat diseases associated with impaired expression of HFE gene, including those due to insufficient expression of HFE gene and/or HFE gene mutations, including haemochromatosis, was developed that involves transfection of the cells of patient or animal organs and tissues with the gene therapy DNA vector carrying the therapeutic gene based on the gene therapy DNA vector GDTT1.8NAS12, and/or injection of autologous cells of a patient or animal, transfected with the selected gene therapy DNA vector carrying the therapeutic gene based on the gene therapy DNA vector GDTT1.8NAS12 into the organs and tissues of the patient or animal, and/or injection of the gene therapy DNA vector carrying the therapeutic gene based on the gene therapy DNA vector GDTT1.8NAS12 into the organs and tissues of the same patient or animal, or the combination of the indicated methods.

A method of production of strains for construction of the gene therapy DNA vector in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis was developed that involves obtaining electrocompetent cells of Escherichia coli strain JM 110-NAS with further electroporation of those cells with the gene therapy DNA vector GDTT1.8NAS12-HFE. After that, the cells are seeded to Petri dishes with a selective medium containing yeastrel, peptone, 6% sucrose, and 10 μg/ml of chloramphenicol, and as a result, Escherichia coli strain JM110-NAS/GDTT1.8NAS12-HFE is obtained.

The Escherichia coli strain JM110-NAS/GDTT1.8NAS12-HFE carrying the gene therapy DNA vector GDTT1.8NAS12-HFE for production thereof allowing for antibiotic-free selection in the process of a gene therapy DNA vector production is claimed for the treatment of diseases associated with function disorder of the HFE protein responsible for regulation of iron metabolism in humans, to treat diseases associated with impaired expression of HFE gene, including those due to insufficient expression of HFE gene and/or HFE gene mutations, including haemochromatosis.

A method of industrial-scale production of the gene therapy DNA vector based on the gene therapy DNA vector GDTT1.8NAS12 carrying the HFE therapeutic gene in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis was developed that involves production of the gene therapy DNA vector GDTT1.8NAS12-HFE by inoculating a culture flask containing the prepared medium with seed culture of the Escherichia coli strain JM110-NAS/GDTT1.8NAS12-HFE, then the cell culture is incubated in an incubator shaker and transferred to an industrial fermenter, then grown to a stationary phase, then the fraction containing the therapeutic DNA product is extracted, multi-stage filtered, and purified by chromatographic methods.

### BRIEF DESCRIPTION OF DRAWINGS

FIG. 1
shows the structure of the gene therapy DNA vector GDTT1.8NAS12 carrying the HFE therapeutic gene that constitutes a circular double-stranded DNA molecule capable of autonomous replication in Escherichia coli cells.

FIG. 1 shows the structure of the DNA vector GDTT1.8NAS12-HFE.

The following structural elements of the vector are indicated in the structures:
EF1a pr—the promoter region of human elongation factor EF1A with an intrinsic enhancer contained in the first intron of the gene. It ensures efficient transcription of the recombinant gene in most human tissues; The reading frame of the therapeutic gene corresponding to the coding region of the HFE gene.
hGH TA—transcription terminator;
pUCori—the origin of replication for autonomous replication with a single nucleotide substitution to increase plasmid production in the cells of most Escherichia coli strains;
RNA-out—the regulatory element RNA-out of transposon Tn 10 that allows for antibiotic-free positive selection in case of the use of Escherichia coli strain JM110NAS.
Unique restriction sites are marked.

FIG. 2
shows diagrams of cDNA amplicon accumulation of the therapeutic gene, namely the HFE gene, in the Hep G2 human hepatocellular carcinoma cell culture (ATCC HB-8065) before its transfection and 48 hours after transfection with the gene therapy DNA vector GDTT1.8NAS12-HFE in order to assess the ability to penetrate into eukaryotic cells and functional activity, i.e. expression of the therapeutic gene at the mRNA level.

FIG. 2 shows curves of accumulation of amplicons during the reaction corresponding to:
1—cDNA of the HFE gene in the Hep G2 cell culture before transfection with the DNA vector GDTT1.8NAS12-HFE,
2—cDNA of the HFE gene in the Hep G2 culture after transfection with the DNA vector GDTT1.8NAS12-HFE,
3—cDNA of the B2M gene in the Hep G2 cell culture before transfection with the DNA vector GDTT1.8NAS12-HFE,
4—cDNA of the B2M gene in the Hep G2 cell culture after transfection with the DNA vector GDTT1.8NAS12-HFE.
B2M (beta-2-microglobuline) gene listed in the GenBank database under number NM 004048.2 was used as a reference gene.

FIG. 3
shows the plot of HFE protein concentration in the cell lysate of the Hep G2 human hepatocellular carcinoma cell culture (ATCC HB-8065) after transfection of these cells with the DNA vector GDTT1.8NAS12-HFE in order to assess the functional activity, i.e. expression at the protein level based on the HFE protein concentration change in the cell lysate.

The following elements are shown in FIG. 3:
culture A—Hep G2 culture transfected with aqueous dendrimer solution without plasmid DNA (reference),
culture B—Hep G2 culture transfected with the DNA vector GDTT1.8NAS12,
culture C—Hep G2 culture transfected with the DNA vector GDTT1.8NAS12-HFE.

FIG. 4
shows the plot of the HFE protein concentration in the skin biopsy samples of three patients after injection of the gene therapy DNA vector GDTT1.8NAS12-HFE into the skin of these patients in order to assess the functional activity, i.e. the expression of the therapeutic gene at the protein level, and the possibility of increasing the level of protein expression using the gene therapy DNA vector based on the gene therapy DNA vector GDTT1.8NAS12 carrying the HFE therapeutic gene.

The following elements are indicated in FIG. 4:
P11 is patient P1 skin biopsy in the region of injection of the gene therapy DNA vector GDTT1.8NAS12-HFE,
P111 is patient P1 skin biopsy in the region of injection of the gene therapy DNA vector GDTT1.8NAS12 (placebo),
P1111 is patient P1 skin biopsy from an intact site,
P21 is patient P2 skin biopsy in the region of injection of the gene therapy DNA vector GDTT1.8NAS12-HFE,
P211 is patient P2 skin biopsy in the region of injection of the gene therapy DNA vector GDTT1.8NAS12 (placebo),
P2111 is patient P2 skin biopsy from an intact site,
P31 is patient P3 skin biopsy in the region of injection of the gene therapy DNA vector GDTT1.8NAS12-HFE,
P311 is patient P3 skin biopsy in the region of injection of the gene therapy DNA vector GDTT1.8NAS12 (placebo),
P3111 is patient P3 skin biopsy from an intact site.

FIG. 5
shows the plot of HFE protein concentration in human skin biopsy samples after intracutaneous injection of autologous fibroblast cell culture transfected with the gene therapy DNA vector GDTT1.8NAS12-HFE in order to demonstrate the method of use by injecting autologous cells transfected with the gene therapy DNA vector GDTT1.8NAS12-HFE.

The following elements are indicated in FIG. 5:
P1C is patient P1 skin biopsy in the region of injection of autologous fibroblast culture of the patient transfected with the gene therapy DNA vector GDTT1.8NAS12-HFE,
P1B is patient P1 skin biopsy in the region of injection of autologous fibroblasts of the patient transfected with the gene therapy DNA vector GDTT1.8NAS12,
P1A is patient P1 skin biopsy from an intact site.

FIG. 6
shows the plot of human HFE protein concentration in the liver tissues of three Wistar rats after intravenous injection of the gene therapy DNA vector GDTT1.8NAS12-HFE in order to demonstrate the method of use of a mixture of gene therapy DNA vectors.

The following elements are indicated in FIG. 6:
K11 is a sample of K1 rat liver tissue upon injection of the gene therapy DNA vector GDTT1.8NAS12-HFE,
K111 is a sample of K1 rat liver tissue upon injection of the gene therapy DNA vector GDTT1.8NAS12 (placebo),
K1111 is a fragment of K1 rat liver tissue from reference intact site (liver),
K21 is a sample of K2 rat liver tissue upon injection of the gene therapy DNA vector GDTT1.8NAS12-HFE,
K211 is a sample of K2 rat liver tissue upon injection of the gene therapy DNA vector GDTT1.8NAS12 (placebo),
K2111 is a fragment of K2 rat liver tissue from reference intact site (liver),
K31 is a sample of K3 rat liver tissue upon injection of the gene therapy DNA vector GDTT1.8NAS12-HFE,
K311 is a sample of K3 rat liver tissue upon injection of the gene therapy DNA vector GDTT1.8NAS12 (placebo),
K3111 is a fragment of K3 rat liver tissue from reference intact site (liver).

FIG. 7
shows diagrams of cDNA amplicon accumulation of the HFE therapeutic gene in bovine peripheral blood mononuclear cells (PBMC) before and 48 hours after transfection of these cells with the DNA vector GDTT1.8NAS12-HFE in order to demonstrate the method of use by injecting the gene therapy DNA vector in animals.

FIG. 7 shows curves of amplicon accumulation during the reaction, corresponding to:
1—cDNA of HFE gene in bovine PBMC cells before transfection with the gene therapy DNA vector GDTT1.8NAS12-HFE,
2—cDNA of HFE gene in bovine PBMC cells after transfection with the gene therapy DNA vector GDTT1.8NAS12-HFE,
3—cDNA of ACT gene in bovine PBMC cells before transfection with the gene therapy DNA vector GDTT1.8NAS12-HFE,
4—cDNA of ACT gene in bovine PBMC cells after transfection with the gene therapy DNA vector GDTT1.8NAS12-HFE.

Bull/cow actin gene (ACT) listed in the GenBank database under number AH001130.2 was used as a reference gene.

### DESCRIPTION OF THE INVENTION

A gene therapy DNA vector carrying human therapeutic gene designed to increase the expression level of this therapeutic gene in human and animal tissues was constructed based on 2591 bp DNA vector GDTT1.8NAS12. The method of production of the gene therapy DNA vector carrying the therapeutic gene is to clone the protein coding sequence of the HFE therapeutic gene (encodes the HFE protein) to the polylinker of the gene therapy DNA vector GDTT1.8NAS12. The ability of DNA vectors to penetrate into eukaryotic cells is known to be due mainly to the vector size. DNA vectors with the smallest size have higher penetration capability. Thus, the absence of elements in the vector that bear no functional load but at the same time increase the vector DNA size is preferred. These features of DNA vectors were taken into account during the production of the gene therapy DNA vectors based on gene therapy DNA vector GDTT1.8NAS12 carrying the HFE therapeutic gene with no large non-functional sequences and antibiotic resistance genes in the vector, which, in addition to technological advantages and safe use, allowed for the significant reduction of size of the produced gene therapy DNA vector GDTT1.8NAS12 carrying the HFE therapeutic gene. Thus, the ability of the obtained gene therapy DNA vector to penetrate into eukaryotic cells is due to its small length.

Gene therapy DNA vector GDTT1.8NAS12-HFE was produced as follows: the coding region of the HFE therapeutic gene was cloned to the gene therapy DNA vector GDTT1.8NAS12, and thus the gene therapy DNA vector GDTT1.8NAS12-HFE, SEQ ID No. 1, was obtained. The coding region of the HFE gene (1051 bp) was produced by isolating total RNA from a biological tissue sample of a healthy human. The reverse transcription reaction was used for the synthesis of the first strand cDNA of the human HFE gene. Amplification was performed using oligonucleotides produced for this purpose by the chemical synthesis method. The amplification product was cleaved by specific restriction endonucleases taking into account the optimal procedure for further cloning, and cloning to the gene therapy DNA vector GDTT1.8NAS12 was performed by KpnI and SalI restriction sites located in the GDTT1.8NAS12 vector polylinker. The selection of restriction sites was carried out in such a way that the cloned fragment entered the reading frame of expression cassette of the vector GDTT1.8NAS12, while the protein coding sequence did not contain restriction sites for the selected endonucleases. Experts in this field realise that the methodological implementation of the gene therapy DNA vector GDTT1.8NAS12-HFE production can vary in terms of selection of known methods of molecular gene cloning, and these methods are included in the scope of this invention. For example, different oligonucleotide sequences can be used to amplify the HFE gene, as well as different restriction endonucleases or laboratory techniques, such as ligation-independent cloning of genes.

The gene therapy DNA vector GDTT1.8NAS12-HFE has the nucleotide sequence SEQ ID No. 1. Also, experts in this field are aware of the degeneracy property of genetic code, and therefore the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of the GDTT1.8NAS12 vector. Also, experts in this field are aware of the genetic polymorphism, and therefore the scope of this invention also includes variants of nucleotide sequences of the HFE gene that also encode different variants of the amino acid sequences of the HFE protein that do not differ from those listed in their functional activity under physiological conditions.

The ability to penetrate into eukaryotic cells and express functional activity, i.e. the ability to express the therapeutic gene of the obtained gene therapy DNA vector GDTT1.8NAS12-HFE is confirmed by injecting the obtained vector into eukaryotic cells and subsequent analysis of the expression of specific mRNA and/or protein product of the therapeutic gene. The presence of specific mRNA in cells into which the gene therapy DNA vector GDTT1.8NAS12-HFE was injected shows the ability of the obtained vector to both penetrate into eukaryotic cells and express mRNA of the therapeutic gene. Furthermore, it is known to the experts in this field that the presence of mRNA gene is a mandatory condition but not an evidence of the translation of protein encoded by the therapeutic gene. Therefore, in order to confirm properties of the gene therapy DNA vector GDTT1.8NAS12-HFE to express the therapeutic gene at the protein level in eukaryotic cells into which the gene therapy DNA vector was injected, analysis of the concentration of protein encoded by the therapeutic gene was carried out using immunological methods. The presence of the HFE protein confirms the efficiency of expression of therapeutic genes in eukaryotic plant cells and the possibility of increasing the protein concentration using the gene therapy DNA vector based on the gene therapy DNA vector GDTT1.8NAS12 carrying the HFE therapeutic gene. Thus, in order to confirm the efficiency of the produced gene therapy DNA vector GDTT1.8NAS12-HFE carrying the therapeutic gene, namely the HFE gene, the following methods were used:
A) real-time PCR, i.e. change in cDNA accumulation of the therapeutic gene in human and animal cell lysate after transfection of different human and animal cell lines with the gene therapy DNA vector,
B) Enzyme-linked immunosorbent assay, i.e. change in the quantitative level of the therapeutic protein in the human cell lysate after transfection of different human cell lines with the gene therapy DNA vector,
C) Enzyme-linked immunosorbent assay, i.e. change in the quantitative level of the therapeutic protein in the supernatant of human and animals tissue biopsy samples after injection of the gene therapy DNA vector into these tissues,
D) Enzyme-linked immunosorbent assay, i.e. change in the quantitative level of the therapeutic proteins in the supernatant of human tissue biopsies after injection of these tissues with autologous cells of this human, transfected with the gene therapy DNA vector.

In order to confirm the practicability of use of the constructed gene therapy DNA vector GDTT1.8NAS12-HFE carrying the therapeutic gene, namely the HFE gene, the following was performed:
A) transfection of various human and animal cell lines with the gene therapy DNA vector,
B) injection of the gene therapy DNA vector into various human and animal tissues,
C) injection of the gene therapy DNA vector into animal tissues,
D) injection of autologous cells transfected with the gene therapy DNA vector into human tissues.

These methods of use are free of potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of the gene therapy DNA vector GDTT1.8NAS12-HFE (SEQ ID No. 1).

It is known to the experts in this field that antibiotic resistance genes in the gene therapy DNA vectors are used to obtain these vectors in preparative quantities by increasing bacterial biomass in a nutrient medium containing a selective antibiotic. Within this invention, in order to ensure the safe use of the gene therapy DNA vector GDTT1.8NAS12 carrying the HFE therapeutic gene, the use of selective nutrient media containing an antibiotic is not possible. A method for obtaining strains for the production of this gene therapy vector on the basis of the Escherichia coli strain JM110-NAS is proposed as a technological solution for obtaining the gene therapy DNA vector GDTT1.8NAS12 carrying the HFE therapeutic gene in order to scale up the production of the gene therapy vectors to an industrial scale. The method of production of the Escherichia coli strain JM110-NAS/GDTT1.8NAS12-HFE involves production of competent cells of the Escherichia coli strain JM110-NAS with the injection of the gene therapy DNA vector GDTT1.8NAS12-HFE into these cells using transformation (electroporation) methods widely known to the experts in this field. The obtained Escherichia coli strain JM110-NAS/GDTT1.8NAS12-HFE is used to produce the gene therapy DNA vector GDTT1.8NAS12-HFE allowing for the use of antibiotic-free media.

In order to confirm the production of the Escherichia coli strain JM110-NAS/GDTT1.8NAS12-HFE transformation, selection, and subsequent biomass growth with extraction of plasmid DNA were performed.

To confirm the producibility and constructability and scale up of the production of the gene therapy DNA vector GDTT1.8NAS12-HFE carrying the therapeutic gene, namely the HFE gene, to an industrial scale, the fermentation on an industrial scale of the Escherichia coli strain Escherichia coli JM110-NAS/GDTT1.8NAS12-HFE containing the gene therapy DNA vector GDTT1.8NAS12 carrying the therapeutic gene, namely HFE, was performed.

The method of scaling the production of bacterial mass to an industrial scale for the isolation of the gene therapy DNA vector GDTT1.8NAS12 carrying the HFE therapeutic gene involves incubation of the seed culture of the Escherichia coli strain JM110-NAS/GDTT1.8NAS12-HFE in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i. e. the gene therapy DNA vector GDTT1.8NAS12-HFE is extracted, multi-stage filtered, and purified by chromatographic methods. It is known to the experts in this field that culture conditions of producer strains, composition of nutrient media (except for absence of antibiotics), equipment used, and DNA purification methods may vary within the standard operating procedures depending on the particular production line, but known approaches to scaling, industrial production, and purification of DNA vectors using the Escherichia coli strain JM110-NAS/GDTT1.8NAS12-HFE fall within the scope of this invention.

The described disclosure of the invention is illustrated by examples of the embodiment of this invention.

The essence of the invention is explained in the following examples.

### Example 1

Production of gene therapy DNA vector GDTT1.8NAS12-HFE carrying the HFE therapeutic gene.

The gene therapy DNA vector GDTT1.8NAS12-HFE was constructed by cloning the coding region of the HFE gene (1051 bp) to the 2591 bp DNA vector GDTT1.8NAS12 by SalI and KpnI restriction sites. The coding region of the HFE gene (1051 bp) was obtained by isolating total RNA from the biological tissue sample from a healthy human, followed by reverse transcription reaction using Mint-2 commercial kit (Evrogen, Russia) and PCR amplification using the following oligonucleotides:

HFE-up
TTTGTCGACCACCATGGGCCCGCGAGCCAGGCCGG,
HFE-lo
AATGGTACCTCACTCACGTTCAGCTAAGACGTAGTGC,

and commercially available kit Phusion® High-Fidelity DNA Polymerase (New England Biolabs, USA).

The gene therapy DNA vector GDTT1.8NAS12 was constructed by consolidating six fragments of DNA derived from different sources:
(a) the origin of replication was produced by PCR amplification of a region of commercially available pUC19 plasmid with UCori-Bam and UCori-Nco oligonucleotides (List of Sequences, (1)-(2)),
(b) hGH-TA transcription terminator was obtained by PCR amplification of a site of human genomic DNA using hGH-F and hGH-R oligonucleotides (List of Sequences, (3) and (4)),
(c) the RNA-OUT regulatory site of transposon Tn10 was synthesised from RO—F, RO—R, RO-1, RO-2, and RO-3 oligonucleotides (List of Sequences, (5)-(9)),
(d) the kanamycin resistance gene was obtained by PCR amplification of a site of commercially available human pET-28 plasmid using Kan-F and Kan-R oligonucleotides (List of Sequences, (10) and (11)),
(e) the polylinker was obtained by radiolabelling and annealing of four synthetic oligonucleotides, MCS1, MCS2, MCS3, and MCS4 (List of Sequences, (12)-(15)),
(f) promoter/regulator region of EF1a human elongation factor gene with its own enhancer was obtained by PCR amplification of a site of human genomic DNA using EF1-Xho and EF1-R oligonucleotides (List of Sequences, (16)-(17)).

PCR amplification was performed using the commercially available kit Phusion® High-Fidelity DNA Polymerase (New England Biolabs) as per the manufacturer's instructions. The fragments (b), (c), and (d) had overlapping regions allowing for their consolidation with subsequent PCR amplification. Fragments (b), (c), and (d) were joined using hGH-F and Kan-R oligonucleotides (List of Sequences, (3) and (11)). Afterwards, the obtained DNA fragments were consolidated by restriction with subsequent ligation by BamHI and NcoI sites. This resulted in a vector still devoid of the polylinker. To add it, the plasmid was split by restriction endonucleases by BamHI and EcoRI sites with subsequent ligation to fragment (e). This resulted in a 2408 bp intermediate vector carrying a kanamycin resistance gene, but still without promoter/regulator site of elongation factor EF1a gene with its own enhancer. The obtained vector was split by restriction endonucleases by SalI and BamHI sites with subsequent ligation to fragment (f). This resulted in a 3608 bp vector carrying a kanamycin resistance gene and promoter/regulator site of elongation factor EF1a gene with its own enhancer. Then the kanamycin resistance gene was cleaved by SpeI restriction sites, and the remaining fragment was ligated to itself. This resulted in a 2591 bp recombinant gene therapy DNA vector GDTT1.8NAS12 enabling antibiotic-free selection and expression of the therapeutic genes cloned into it in the most types of human and animal tissues.

The amplification product of the coding region of the HFE gene and the DNA vector GDTT1.8NAS12 was cleaved by SalI and KpnI restriction endonucleases (New England Biolabs, USA).

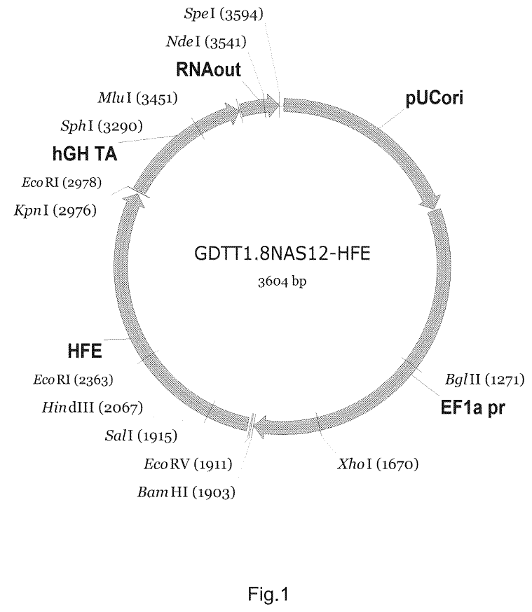

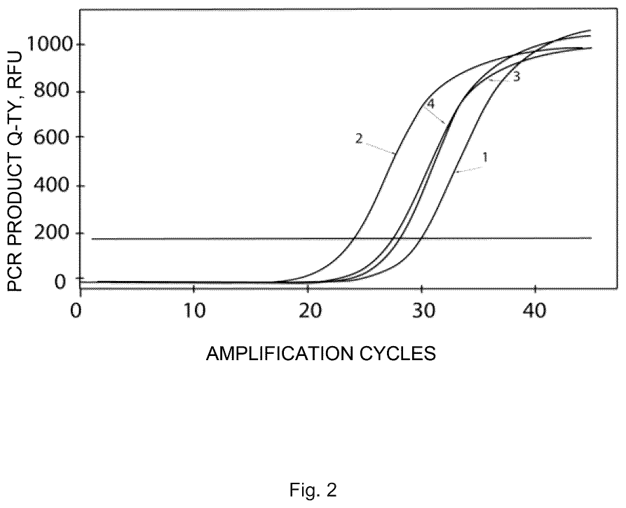

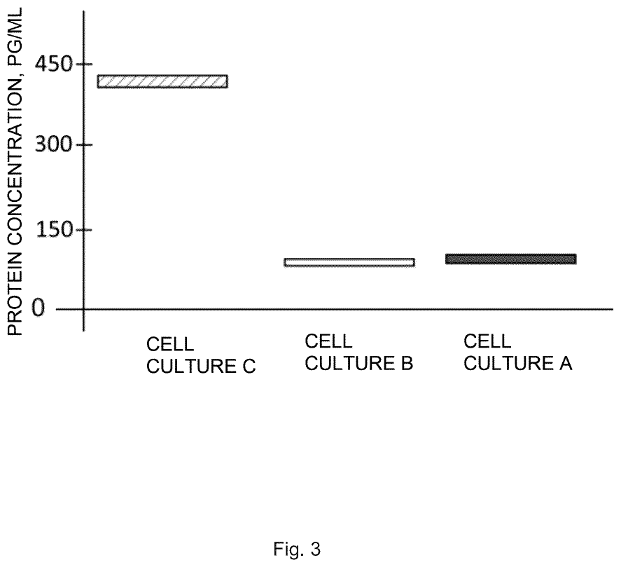

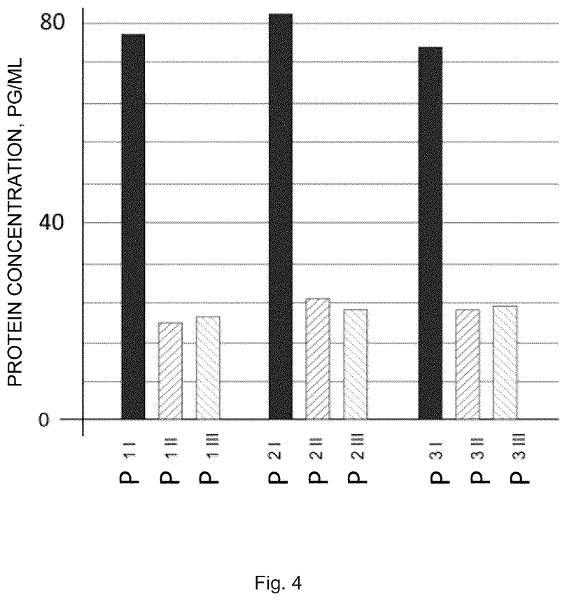

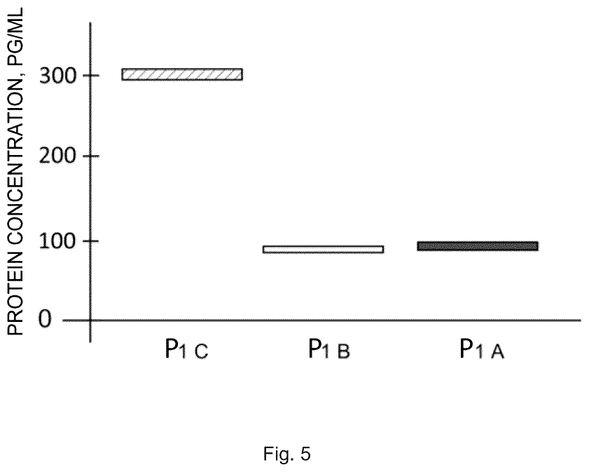

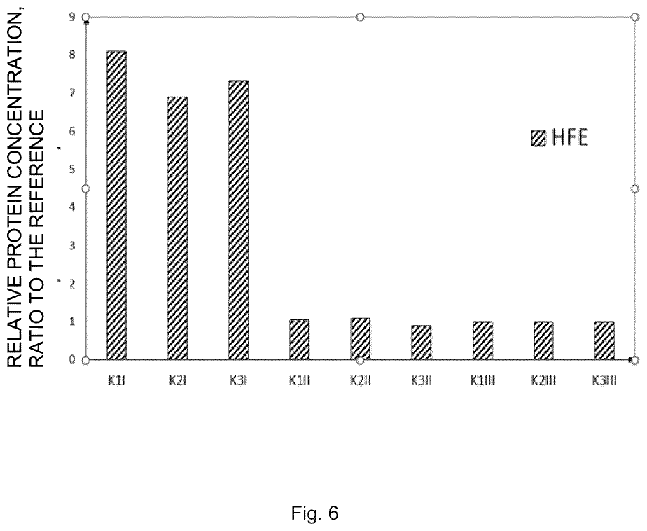

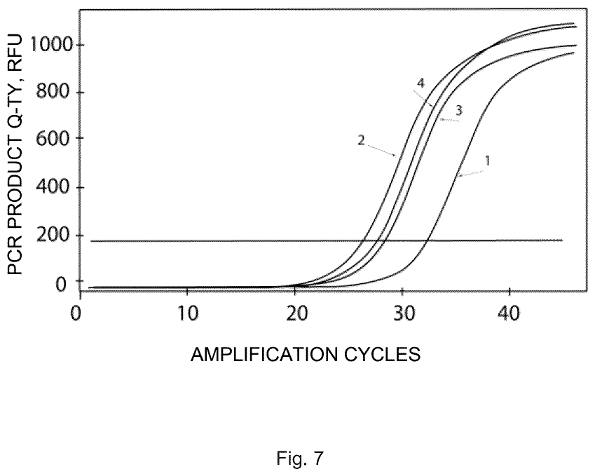

This resulted in the 3604 bp DNA vector GDTT1.8NAS12-HFE with the nucleotide sequence SEQ ID No. 1 and general structure shown in FIG. 1.

### Example 2

Proof of the ability of the gene therapy DNA vector GDTT1.8NAS12-HFE carrying the therapeutic gene, namely the HFE gene, to penetrate eukaryotic cells and its functional activity at the level of therapeutic gene mRNA expression. This example also demonstrates practicability of the use of the gene therapy DNA vector carrying the therapeutic gene.

Changes in the mRNA accumulation of the HFE therapeutic gene were assessed in Hep G2 human hepatocellular carcinoma cell culture (ATCC HB-8065) 48 hours after transfection thereof with the gene therapy DNA vector GDTT1.8NAS12-HFE carrying the human HFE gene. The amount of mRNA was determined by the dynamics of accumulation of cDNA amplicons in the real-time PCR.

The Hep G2 cell culture was grown under standard conditions (37° C., 5% CO2) using the DMEM growth medium with the addition of 10% fetal bovine serum (Gibco). The growth medium was replaced every 48 hours during the cultivation process.

To achieve 90% confluence, 24 hours before the transfection procedure, the cells were seeded into a 24-well plate in the quantity of 5×104 cells per well. Transfection with the gene therapy DNA vector GDTT1.8NAS12-HFE expressing the human HFE gene was performed using Lipofectamine 3000 (ThermoFisher Scientific, USA) according to the manufacturer's recommendations. In the test tube 1, 1 μl of the DNA vector GDTT1.8NAS12-HFE solution (in concentration of 500 ng/μl) and 1 μl of P3000 reagent was added to 25.1 μl of the Opti-MEM medium (Gibco, USA). The preparation was mixed by gentle shaking. In the test tube 2, 1.1 μl of Lipofectamine 3000 solution was added to 25 μl of Opti-MEM medium (Gibco, USA). The preparation was mixed by gentle shaking. The contents of test tube 1 were added to the contents of test tube 2, and the mixture was incubated at room temperature for 5 minutes. The resulting solution was added dropwise to the cells in the volume of 40 μl.

Hep G2 cells transfected with the gene therapy DNA vector GDTT1.8NAS12 devoid of the inserted therapeutic gene (cDNA of the HFE gene before and after transfection with the gene therapy DNA vector GDTT1.8NAS12 devoid of the inserted therapeutic gene is not shown in the figures) were used as a reference. The reference vector GDTT1.8NAS12 for transfection was prepared as described above.

Total RNA was extracted from Hep G2 cells using Trizol Reagent (Invitrogen, USA) according to the manufacturer's recommendations. 1 ml of Trizol Reagent was added to the well with cells, homogenised and heated for 5 minutes at 65° C. Then the sample was centrifuged at 14,000 g for 10 minutes and heated again for 10 minutes at 65° C. Then 2001 μl of chloroform was added, and the mixture was gently mixed and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The packed cells were rinsed in 1 ml of 70% ethyl alcohol, air-dried, and dissolved in 10 μl of RNase-free water. The level of the HFE gene mRNA expression after transfection was determined by assessing the dynamics of cDNA amplicon accumulation by real-time PCR. For production and amplification of cDNA specific for the human HFE gene, the following HFE_SF and HFE_SR oligonucleotides were used:

HFE_SF
TGATCATGAGAGTCGCCGTG,
HFE_SR
TTTCACAGCCCAGGATGACC.

The length of amplification product is 194 bp.

Reverse transcription reaction and PCR amplification was performed using SYBR GreenQuantitect RT-PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles of denaturation at 94° C. for 15 seconds, the annealing of primers at 60° C. for 30 seconds, and elongation at 72° C. for 30 seconds. B2M (beta-2-microglobuline) gene listed in the GenBank database under number NM 004048.2 was used as a reference gene. Positive control included amplicons from PCR on matrices formed by plasmids in known concentrations containing cDNA sequences of the HFE and B2M genes. Deionised water was used as negative control. Real-time quantification of the cDNA amplicons of the HFE and B2M genes and assessment of dynamics thereof was conducted using the Bio-Rad CFX Manager 2.1 software (Bio-Rad, USA). Diagrams resulting from the assay are shown in FIG. 2.

FIG. 2 shows that the level of specific mRNA of the human HFE gene has grown massively as a result of transfection of the Hep G2 human hepatocellular carcinoma cell culture (ATCC HB-8065) with the gene therapy DNA vector GDTT1.8NAS12-HFE, which confirms the ability of the vector to penetrate eukaryotic cells and express the HFE gene at the mRNA level. The presented results also confirm the practicability of use of the gene therapy DNA vector GDTT1.8NAS12-HFE in order to increase the expression level of the HFE gene in eukaryotic cells.

### Example 3

Proof of the efficiency and practicability of use of the gene therapy DNA vector GDTT1.8NAS12-HFE carrying the HFE gene in order to increase the expression of the HFE protein in mammalian cells.

The change in the HFE protein concentration in the Hep G2 human hepatocellular carcinoma cell culture (ATCC HB-8065) was assessed after transfection of the culture with the DNA vector GDTT1.8NAS12-HFE carrying the human HFE gene.

The Hep G2 human hepatocellular carcinoma cell culture was cultivated as per Example 2.

To achieve 90% confluence, 24 hours before the transfection procedure, the cells were seeded into a 24-well plate in the quantity of 5×104 cells per well. The 6th generation SuperFect Transfection Reagent (Qiagen, Germany) was used for transfection. The aqueous dendrimer solution without DNA vector (A) and DNA vector GDTT1.8NAS12 devoid of cDNA of the HFE gene (B) were used as a reference, and the DNA vector GDTT1.8NAS12-HFE carrying the human HFE gene (C) was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, the culture medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 μg/ml of gentamicin was added and incubated for 24-48 hours at 37° C. in the presence of 5% CO2.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and mixed thoroughly. Supernatant was collected and used to assay the therapeutic protein. The HFE protein was assayed by enzyme-linked immunosorbent assay (ELISA) using the HFE ELISA Kit (Haemochromatosis) (ABIN815944, https://www.antibodies-online.com/kit/815944/Haemochromatosis+HFE+ELISA+Kit/) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of the HFE protein was used. The sensitivity was at least 78 μg/ml, with measurement range from 78 μg/ml to 5000 μg/ml. R-3.0.2 software (https://www.r-project.org/) was used for the statistical treatment of the results and data visualization. Diagrams resulting from the assay are shown in FIG. 3.

FIG. 3 shows that the transfection of the Hep G2 human hepatocellular carcinoma cell culture with the gene therapy DNA vector GDTT1.8NAS12-HFE results in increased HFE protein concentration compared to reference samples, which confirms the ability of the vector to penetrate eukaryotic cells and express the HFE gene at the protein level. The presented results also confirm the practicability of use of the gene therapy DNA vector GDTT1.8NAS12-HFE to enhance the expression level of the HFE gene in eukaryotic cells.

### Example 4

Proof of the efficiency and practicability of use of the gene therapy DNA vector GDTT1.8NAS12-HFE carrying the HFE gene in order to increase the expression of the HFE protein in human cells.

To confirm the efficiency of the gene therapy DNA vector GDTT1.8NAS12-HFE carrying the therapeutic gene, namely the HFE gene, and practicability of its use, changes in the HFE protein concentration in human skin upon intracutaneous injection of the gene therapy DNA vector GDTT1.8NAS12-HFE carrying the human HFE gene were assessed.

To analyse changes in the HFE protein concentration, the gene therapy DNA vector GDTT1.8NAS12-HFE carrying the HFE gene was injected into the forearm skin of three patients with concurrent injection of a placebo, the gene therapy DNA vector GDTT1.8NAS12 devoid of cDNA of the HFE gene.

Patient 1, male, 63 y.o. (P1); patient 2, female, 64 y.o. (P2); patient 3, male, 69 y.o. (P3). Transfection reagent cGMP grade in-vivo-jetPEI (Polyplus Transfection, France) was used as a transport system. The gene therapy DNA vector GDTT1.8NAS12-HFE containing cDNA of the HFE gene, and the gene therapy DNA vector GDTT1.8NAS12 used as a placebo and devoid of cDNA of the HFE gene were dissolved in sterile nuclease-free water. To obtain a gene construct, DNA-cGMP grade in-vivo-jetPEI complexes were prepared according to the manufacturer recommendations.

The gene therapy DNA vector GDTT1.8NAS12 (placebo) and the gene therapy DNA vector GDTT1.8NAS12-HFE carrying the HFE gene were injected in the quantity of 1 mg of each genetic construct using the tunnel method with a 30G needle to the depth of 3 mm. The injectate volume of the gene therapy DNA vector GDTT1.8NAS12 (placebo) and the gene therapy DNA vector GDTT1.8NAS12-HFE carrying the HFE gene was 0.5 ml for each genetic construct. The points of injection of each genetic construct were located at 8 to 10 cm intervals at the forearm site.

Biopsy samples were taken on the 2nd day after the injection of the genetic constructs of the gene therapy DNA vectors. The biopsy samples were taken from the patients' skin in the site of injection of the gene therapy DNA vector GDTT1.8NAS12-HFE carrying the HFE gene (1), the gene therapy DNA vector GDTT1.8NAS12 (placebo) (II), and from intact skin (111), using the skin biopsy device Epitheasy 3.5 (Medax SRL, Italy). The skin of patients in the biopsy site was preliminarily rinsed with sterile saline and anaesthetised with a lidocaine solution. The biopsy sample had a size of ca. 10 mm3 and weight ca. 11 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used in order to assay the therapeutic protein by enzyme-linked immunosorbent assay (ELISA). The HFE protein was assayed by enzyme-linked immunosorbent assay (ELISA) as described in Example 3.

To measure the numerical value of concentration, the calibration curve constructed using the reference samples from the kit with known concentrations of the HFE protein was used. R-3.0.2 software (https://www.r-project.org/) was used for the statistical treatment of the results and data visualization according to the manufacturer's method with optical density detection using ChemWell Automated EIA and Chemistry Analyser (Awareness Technology Inc., USA). Diagrams resulting from the assay are shown in FIG. 4.

FIG. 4 shows the increased HFE protein concentration in the skin of all three patients in the injection site of the gene therapy DNA vector GDTT1.8NAS12-HFE carrying the human HFE therapeutic gene, compared to the HFE protein concentration in the injection site of the gene therapy DNA vector GDTT1.8NAS12 (placebo) devoid of the human HFE gene, which indicates the efficiency of the gene therapy DNA vector GDTT1.8NAS12-HFE and confirms the practicability of its use, in particular upon intracutaneous injection of the gene therapy DNA vector in human tissues.

### Example 5

Proof of efficiency of the gene therapy DNA vector GDTT1.8NAS12-HFE carrying the HFE gene and practicability of its use in order to increase the expression level of the HFE protein in human tissues by injecting autologous fibroblasts transfected with the gene therapy DNA vector GDTT1.8NAS12-HFE.

To confirm the efficiency of the gene therapy DNA vector GDTT1.8NAS12-HFE carrying the HFE gene and practicability of its use, changes in the HFE protein concentration in human skin upon intracutaneous injection of autologous fibroblast culture of the same patient, transfected with the gene therapy DNA vector GDTT1.8NAS12-HFE were assessed.

The appropriate autologous fibroblast culture transfected with the gene therapy DNA vector GDTT1.8NAS12-HFE carrying the HFE gene was injected into the patient's forearm skin with concurrent injection of a placebo in the form of autologous fibroblast culture transfected with the gene therapy DNA vector GDTT1.8NAS12 devoid of the HFE gene.

The human primary fibroblast culture was isolated from the patient skin biopsy specimens. Biopsy specimens of the skin from the area protected from ultraviolet, namely behind the ear or on the inner lateral side of the elbow, were taken using the skin biopsy device Epitheasy 3.5 (Medax SRL, Italy). The biopsy sample size was ca. 10 mm, its weight ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×104 cells was taken from the cell culture. The patient's fibroblast culture was transfected with the gene therapy DNA vector GDTT1.8NAS12-HFE carrying the HFE gene or placebo, the GDTT1.8NAS12 vector devoid of the HFE therapeutic gene.

The transfection was carried out using a cationic polymer, polyethyleneimine JETPEI (Polyplus transfection, France), according to the manufacturer's instructions. The cells were cultured for 72 hours and then injected into the patient. Injection of autologous fibroblast culture of the patient transfected with the gene therapy DNA vector GDTT1.8NAS12-HFE and autologous fibroblast culture of the patient transfected with the gene therapy DNA vector GDTT1.8NAS12 as a placebo was performed in the forearm using the tunnel method with a 13 mm long 30G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after the injection of autologous fibroblast culture transfected with the gene therapy DNA vector GDTT1.8NAS12-HFE carrying the therapeutic gene, namely the HFE gene, and placebo. Biopsy was taken from the patient's skin in the site of injection of autologous fibroblast culture transfected with the gene therapy DNA vector GDTT1.8NAS12-HFE carrying the therapeutic gene, namely the HFE gene (C), autologous fibroblast culture transfected with the gene therapy DNA vector GDTT1.8NAS12 devoid of the HFE therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample had a size of ca. 10 mm3 and weight ca. 11 mg. The sample was placed in a buffer solution containing 50 mmol of Tris-HCl with pH 7.6, 100 mmol of NaCl, 1 mmol of EDTA, and 1 mmol of phenylmethylsulfonyl fluoride, and homogenized to a homogeneous suspension state. The suspension obtained was then centrifuged for 10 minutes at 14,000 g. Supernatant was collected and used to assay the therapeutic protein as described in Example 3 (HFE protein assay).

Diagrams resulting from the assay are shown in FIG. 5.

FIG. 5 shows the increased concentration of the HFE protein in the area of the patient's skin in the injection site of autologous fibroblast culture transfected with the gene therapy DNA vector GDTT1.8NAS12-HFE carrying the HFE gene, compared to the HFE protein concentration in the injection site of autologous fibroblast culture transfected with the gene therapy DNA vector GDTT1.8NAS12 devoid of the HFE gene (placebo), which indicates the efficiency of the gene therapy DNA vector GDTT1.8NAS12-HFE and practicability of its use in order to increase the expression level of the HFE in human tissues, in particular upon injection of autologous fibroblasts transfected with the gene therapy DNA vector GDTT1.8NAS12-HFE.

### Example 6

Proof of the efficiency and practicability of use of the gene therapy DNA vector GDTT1.8NAS12-HFE carrying the HFE gene in order to increase the expression of the HFE protein in mammalian cells.

Changes in the HFE protein concentration in rat liver tissue were assessed upon injection of the gene therapy DNA vector GDTT1.8NAS12-HFE. The study involved 9 laboratory animals, namely male Wistar rats at 8 months of age weighing 240-290 g, divided into three groups: group I for introduction of the gene therapy DNA vector carrying the HFE gene, group II for introduction of the gene therapy DNA vector GDTT1.8NAS12 devoid of the HFE gene (placebo), and group III not subjected to any manipulations. Transfection reagent cGMP grade in-vivo-jetPEI (Polyplus Transfection, France) was used as a transport system. Equimolar mixture of the gene therapy DNA vectors was dissolved in sterile nuclease-free water. To obtain a gene construct, DNA-cGMP grade in-vivo-jetPEI complexes were prepared according to the manufacturer recommendations.

The injectate volume was 1 ml with a total quantity of DNA equal to 500 μg. The solution was injected into the tail vein as a bolus. Rats were decapitated in 2 days after the procedure.

Biopsy was taken from liver (a 50 mg sample) in group I where the gene therapy DNA vector carrying the HFE gene was introduced (samples 1-1, 2-1, 3-1), from liver in group II where the gene therapy DNA vector GDTT1.8NAS12 devoid of the HFE gene (placebo) was introduced (samples 1-II, 2-II, 3-II), and from liver in group III not subjected to any manipulations (samples 1-III, 2-III, 3-III). Each sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension obtained was then centrifuged for 10 minutes at 14,000 g. Supernatant was collected and used to assay the therapeutic protein as described in Example 3 (HFE protein assay). Diagrams resulting from the assay are shown in FIG. 6.

FIG. 6 shows that liver samples from the group of animals treated with the gene therapy DNA vector GDTT1.8NAS12-HFE carrying the therapeutic HFE gene contains much more HFE protein than the liver samples from animals treated with the placebo or from intact animals. The obtained results show the efficiency of the gene therapy DNA vector and confirm practicability of its use to enhance expression of the therapeutic protein in mammalian tissues.

### Example 7

Proof of the efficiency of the gene therapy DNA vector GDTT1.8NAS12-HFE carrying the HFE gene and practicability of its use in order to increase the expression level of the HFE protein in mammalian cells.

To confirm the efficiency of the gene therapy DNA vector GDTT1.8NAS12-HFE carrying the HFE gene, changes in mRNA accumulation of the HFE therapeutic gene in bovine peripheral blood mononuclear cells 48 hours after transfection thereof with the gene therapy DNA vector GDTT1.8NAS12-HFE carrying the human HFE gene were assessed.

Bovine peripheral blood mononuclear cells were extracted from 50 ml of venous blood by centrifuging in the gradient of ficoll solution 1.077 (Paneco, P052p) and cultured in RPMI Media 1640 (GIBCO®, 11875-085) with the addition of 10% of horse serum (ATCC® 30-2040™) under standard conditions. Transfection with the gene therapy DNA vector GDTT1.8NAS12-HFE carrying the human HFE gene and DNA vector GDTT1.8NAS12 not carrying the human HFE gene (reference), RNA extraction, reverse transcription reaction, PCR amplification, and data analysis were performed as described in Example 2. Bull/cow actin gene (ACT) listed in the GenBank database under number AH001130.2 was used as a reference gene. Positive control included amplicons from PCR on matrices represented by plasmids in known concentrations containing the HFE and ACT gene sequences. Deionised water was used as negative control. Real-time quantification of the PCR products, i.e. the HFE and ACT gene cDNAs obtained by amplification, was performed using the Bio-Rad CFX Manager 2.1 software (Bio-Rad, USA).

Diagrams resulting from the assay are shown in FIG. 7.

FIG. 7 shows that the level of specific mRNA of the human HFE gene has grown massively as a result of transfection of bovine peripheral blood mononuclear cells with the gene therapy DNA vector GDTT1.8NAS12-HFE, which confirms the ability of the vector to penetrate eukaryotic cells and express the HFE gene at the mRNA level. The presented results confirm the practicability of use of the gene therapy DNA vector GDTT1.8NAS12-HFE for enhancement of the HFE gene expression in mammalian cells.

### Example 8

The Escherichia coli strain JM110-NAS/GDTT1.8NAS12-HFE carrying the gene therapy DNA vector and method of production thereof.

The strain construction for the production of the gene therapy DNA vector based on the gene therapy DNA vector GDTT1.8NAS12 carrying the HFE therapeutic gene on an industrial scale, namely, the Escherichia coli strain JM110-NAS/GDTT1.8NAS12-HFE allowing for antibiotic-free selection involves making electrocompetent cells of the Escherichia coli strain JM110-NAS and subjecting these cells to electroporation with the gene therapy DNA vector GDTT1.8NAS12-HFE. After that, the cells were poured into agar plates (Petri dishes) with a selective medium containing yeastrel, peptone, 6% sucrose, and 10 μg/ml of chloramphenicol. At the same time, production of the Escherichia coli strain JM110-NAS for the production of the gene therapy DNA vector GDTT1.8NAS12 or the gene therapy DNA vectors based on it, allowing for antibiotic-free positive selection, involved constructing a 64 bp linear DNA fragment that contains regulatory element RNA-IN of Tn10 transposon allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

The obtained strains for production were included in the collection of the National Biological Resource Centre—Russian National Collection of Industrial Microorganisms (NBRC RNCIM), Russia, and NCIMB Patent Deposit Service, UK, under the following registration numbers:

Escherichia coli strain JM110-NAS/GDTT1.8NAS12-HFE under Reg. No. B-13538, deposited on 27.11.2019; INTERNATIONAL DEPOSITARY AUTHORITY No. NCIMB—43522, deposited on 28.11.2019.

### Example 9

The method for scaling up the production of the gene therapy DNA vector based on the gene therapy DNA vector GDTT1.8NAS12 carrying the HFE therapeutic gene to an industrial scale.

To confirm the producibility and constructability of the gene therapy DNA vector GDTT1.8NAS12-HFE (SEQ ID No. 1) on an industrial scale, large-scale fermentation of the Escherichia coli strain JM110-NAS/GDTT1.8NAS12-HFE containing the gene therapy DNA vector GDTT1.8NAS12 carrying the therapeutic gene, namely the HFE gene, was performed. The Escherichia coli strain JM110-NAS/GDTT1.8NAS12-HFE was produced on the basis of the Escherichia coli strain JM110-NAS (Genetic Diagnostics and Therapy 21 Ltd, United Kingdom) as per Example 8 by electroporation of competent cells of this strain with the gene therapy DNA vector GDTT1.8NAS12-HFE carrying the HFE therapeutic gene, with subsequent inoculation of transformed cells into agar plates (Petri dishes) with a selective medium containing yeastrel, peptone, and 6% sucrose, and selection of individual clones.

Fermentation of the resulting Escherichia coli strain JM110-NAS/GDTT1.8NAS12-HFE carrying the gene therapy DNA vector GDTT1.8NAS12-HFE was performed in a 10 L fermenter with subsequent extraction of the gene therapy DNA vector GDTT1.8NAS12-HFE.

For the fermentation of the Escherichia coli strain JM110-NAS/GDTT1.8NAS12-HFE, medium containing the following ingredients per 10 L of volume was prepared: 100 g of tryptone and 50 g of yeastrel (Becton Dickinson, USA); then the medium was diluted with water to 8,800 ml and autoclaved at 121° C. for 20 minutes, and then 1,200 ml of 50% (w/v) sucrose was added. After that, the seed culture of the Escherichia coli strain JM110-NAS/GDTT1.8NAS12-HFE was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were precipitated by centrifugation for 30 minutes at 5,000-10,000 g. Supernatant was removed, and the cell precipitate was re-suspended in 10% (v/v) phosphate buffered saline. The cells were centrifuged again for 30 minutes at 5,000-10,000 g. Supernatant was removed, a solution of 20 mM TrisCl, 1 mM EDTA, 200 g/I sucrose, pH 8.0 was added to the cell precipitate in the volume of 1000 ml, and the mixture was mixed thoroughly to a homogenised suspension. Then egg lysozyme solution was added to the final concentration of 100 μg/ml. The mixture was incubated for 20 minutes on ice while mixing gently. Then 2500 ml of 0.2M NaOH, 10 g/I sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution, and then the gene therapy DNA vector GDTT1.8NAS12-HFE was eluted using a linear gradient of 25 mM Tris-HCl, pH 7.0, to obtain a solution of 25 mM Tris-HCl, pH 7.0, 1M NaCl, five times the volume of the column. The elution process was controlled by measuring optical density of the run-off solution at 260 nm. Chromatographic fractions containing the gene therapy DNA vector GDTT1.8NAS12-HFE were joined together and subjected to gel filtration by Superdex 200 sorbent (GE, USA). The column was equilibrated with phosphate buffered saline. The elution process was controlled by measuring optical density of the run-off solution at 260 nm, and the fractions were analysed by agarose gel electrophoresis. The fractions containing the gene therapy DNA vector GDTT1.8NAS12-HFE were joined together and stored at −20° C. To assess the process reproducibility, the indicated processing operations were repeated five times.

The process reproducibility and quantitative characteristics of the final product yield confirm the producibility and constructability of the gene therapy DNA vector GDTT1.8NAS12-HFE on an industrial scale.

Thus, the produced gene therapy DNA vector containing the therapeutic gene can be used to deliver it to human and animal cells of subjects with reduced or insufficient expression of the functional protein encoded by this gene, or with mutations in the therapeutic gene resulting in expression of non-functional protein, thus ensuring the desired therapeutic effect.

The purpose set in this invention is achieved, namely the construction of the gene therapy DNA vector enhancing expression level of the HFE gene combining the following properties:
I) the effectiveness of increase of the expression of the therapeutic gene in eukaryotic cells due to the obtained gene therapy vectors with a minimum length,
II) possibility of safe use in gene therapy of human beings and animals due to the absence of regulatory elements representing the nucleotide sequences of viral genomes and antibiotic resistance genes in the gene therapy DNA vector,
III) producibility and constructability in the strains on an industrial scale,
IV) as well as the purpose of the constructions of a strain carrying this gene therapy DNA vector for the production of this gene therapy DNA vector is achieved.

This is supported by the following examples:
Examples 1, 2, 3, 4, 5, 6, 7 for Clause 1,
Examples 1, 2, 3, 4, 5, 6, 7 for Clause 11,
Examples 8, 9 for Clauses III and IV.

### INDUSTRIAL APPLICABILITY

All the examples listed above confirm industrial applicability of the proposed gene therapy DNA vector based on the gene therapy DNA vector GDTT1.8NAS12 carrying the HFE therapeutic gene in order to increase the expression level of this therapeutic gene, the Escherichia coli strain JM110-NAS/GDTT1.8NAS12-HFE carrying the gene therapy DNA vector, method of the gene therapy DNA vector production, and method of the gene therapy DNA vector production on an industrial scale.

### List of Abbreviations

GDTT1.8NAS12—a gene therapy vector devoid of sequences of viral genomes and antibiotic resistance markers (vector therapeutic virus-antibiotic-free)
DNA—deoxyribonucleic acid
cDNA—complementary deoxyribonucleic acid
RNA—ribonucleic acid
mRNA—messenger ribonucleic acid
bp—base pair
PCR—polymerase chain reaction
ml—millilitre, μl—microlitre
mm3—cubic millimetre
l—litre
μg—microgram
mg—milligram
g—gram
μmol—micromol
mM—millimol
min—minute
s—second
rpm—rotations per minute
nm—nanometre
cm—centimetre
mW—milliwatt
RFU—relative fluorescence unit
PBS—phosphate buffered saline
PBMC—peripheral blood mononuclear cells.

1. Gene therapy DNA vector based on the gene therapy DNA vector GDTT1.8NAS12 in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis, while the gene therapy DNA vector contains the coding region of the HFE therapeutic gene cloned into the gene therapy DNA vector GDTT1.8NAS12 resulting in the gene therapy DNA vector GDTT1.8NAS12-HFE that has nucleotide sequence SEQ ID No. 1.

2. Gene therapy DNA vector based on the gene therapy DNA vector GDTT1.8NAS12 carrying the HFE therapeutic gene as per CI. 1, wherein the constructed gene therapy DNA vector GDTT1.8NAS12-HFE as per CI. 1 has the ability to efficiently penetrate into human and animal cells due to the limited size of GDTT1.8NAS12 vector part not exceeding 2600 bp and express the HFE therapeutic gene cloned to it.

3. Gene therapy DNA vector based on the gene therapy DNA vector GDTT1.8NAS12 containing the coding region of the HFE therapeutic gene as per CI. 1, wherein the constructed gene therapy DNA vector GDTT1.8NAS12-HFE as per CI. 1 is comprised of nucleotide sequences that don't constitute antibiotic resistance genes, viral genes, or elements of viral genomes, which ensures its safe use in gene therapy of humans and animals.

4. A method of production of the gene therapy DNA vector based on the gene therapy DNA vector GDTT1.8NAS12 carrying the therapeutic gene HFE as per CI. 1, that involves obtaining the gene therapy DNA vector GDTT1.8NAS12-HFE as follows: the coding region of the HFE therapeutic gene is cloned to the gene therapy DNA vector GDTT1.8NAS12, and the gene therapy DNA vector GDTT1.8NAS12-HFE, SEQ ID No. 1, is obtained, while the coding region of the HFE therapeutic gene is obtained by isolating total RNA from a biological human tissue sample followed by the reverse transcription reaction and PCR amplification using the obtained oligonucleotides and cleaving the amplification product by corresponding restriction endonucleases, while cloning to the gene therapy DNA vector GDTT1.8NAS12 is performed by SalI and KpnI restriction sites, while the selection is performed without antibiotics, while the following oligonucleotides produced for this purpose are used as dedicated oligonucleotides for reverse transcription and PCR amplification in the process of production of the gene therapy DNA vector GDTT1.8NAS12-HFE, SEQ ID No. 1:
HFE-up TTTGTCGACCACCATGGGCCCGCGAGCCAGGCCGG,
HFE-lo AATGGTACCTCACTCACGTTCAGCTAAGACGTAGTGC,
and the cleaving of the amplification product and cloning of the coding region of the HFE gene to the gene therapy DNA vector GDTT1.8NAS12 is performed with using SalI and KpnI restriction endonucleases.

5. A method of use of the gene therapy DNA vector based on the gene therapy DNA vector GDTT1.8NAS12 carrying the HFE therapeutic gene as per CI. 1 in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis, that involves transfection of the cells of patient or animal organs and tissues with the gene therapy DNA vector carrying the therapeutic gene based on the gene therapy DNA vector GDTT1.8NAS12, and/or injection of autologous cells of the patient or animal, transfected with the selected gene therapy DNA vector carrying the therapeutic gene based on the gene therapy DNA vector GDTT1.8NAS12 into the organs and tissues of the patient or animal, and/or injection of the gene therapy DNA vector carrying the therapeutic gene based on the gene therapy DNA vector GDTT1.8NAS12 into the organs and tissues of the same patient or animal, or a combination of the indicated methods.

6. A method of production of strains for construction of the gene therapy DNA vector as per CI. 1 in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis, that involves obtaining electrocompetent cells of the Escherichia coli strain JM 110-NAS with subsequent electroporation of those cells with the gene therapy DNA vector GDTT1.8NAS12-HFE. After that, the cells are seeded to Petri dishes with a selective medium containing yeastrel, peptone, 6% sucrose, and 10 μg/ml of chloramphenicol, and as a result, Escherichia coli strain JM110-NAS/GDTT1.8NAS12-HFE is obtained.

7. The Escherichia coli strain JM110-NAS/GDTT1.8NAS12-HFE obtained as per CI. 6 and carrying the gene therapy DNA vector GDTT1.8NAS12-HFE for production thereof allowing for antibiotic-free selection during the production of the gene therapy DNA vector production in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis.

8. A method of production on an industrial scale of the gene therapy DNA vector GDTT1.8NAS12 carrying the HFE therapeutic gene as per CI. 1 in order to treat diseases associated with disorders of functions of HFE protein responsible for regulation of iron metabolism in humans, to treat diseases associated with impaired expression of HFE gene, including those due to insufficient expression of HFE gene and/or HFE gene mutations, including haemochromatosis, that involves production of the gene therapy DNA vector GDTT1.8NAS12-HFE by inoculating a culture flask containing the prepared medium with seed culture of the Escherichia coli strain JM110-NAS/GDTT1.8NAS12-HFE, then the cell culture is incubated in an incubator shaker and transferred to an industrial fermenter, then the fraction containing the target DNA product is extracted, multi-stage filtered, and purified by chromatographic methods.
